(19) United States
(12) Patent Application Publication
(10) Pub. No.: US 2005/0038065 A1
(43) Pub. Date: Feb. 17, 2005

(54) Process for the preparation of 1,5-naphthyridine-3-carboxyamides by direct ester amidation

(75) Inventors: Gregory L. Karrick, Zeeland, MI (US); David H. B. Ripin, Westbrook, CT (US); Lulin Wei, Salem, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/856,161

(22) Filed: May 28, 2004

Related U.S. Application Data
(60) Provisional application No. 60/473,731, filed on May 28, 2003.

(51) Int. Cl. C07D 471/02; A61K 31/4745

(52) U.S. Cl. 514/300; 546/123

(57) ABSTRACT

A new route for the preparation of substituted 1,5-naphthyridine-3-carboxyamides, useful in the diagnosis and treatment of anxiety, Downs Syndrome, sleep, cognitive and seizure disorders, and overdose with benzodiazepine drugs and for enhancement of alertness, is provided. These compounds may be readily prepared by heating the corresponding 1,5-naphthyridine-3-carboxylic acid ester with a primary amine in a polar solvent such as dimethylformamide or dimethylsulfoxide.

### BACKGROUND OF THE INVENTION

This invention relates to a new route for the preparation and purification of substituted 1,5-naphthyridine-3-carboxyamides and the pharmaceutically acceptable non-toxic salts thereof. These compounds are highly selective agonists, antagonists or inverse agonists for GABAa brain receptors or prodrugs of agonists, antagonists or inverse agonists for GABAa brain receptors. These compounds are useful in the diagnosis and treatment of anxiety, Down Syndrome, sleep, cognitive and seizure disorders, and overdose with benzodiazepine drugs and for enhancement of alertness.

The substituted 1,5-naphthyridine-3-carboxyamides that are prepared in accord with the process of the present invention are disclosed in U.S. Pat. No. 6,143,760 and PCT International Publication No. WO99/10347 A1, each of which is incorporated herein by reference in its entirety.

### SUMMARY OF THE INVENTION

The present invention provides a process of preparing a compound of the following formula 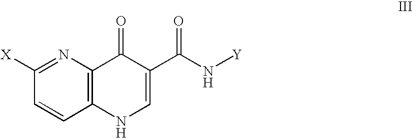
wherein X is hydrogen, halogen, —OR₁, C₁-C₆ alkyl optionally substituted with up to three groups selected independently from halogen and hydroxy, or —NR₂R₃; phenyl, naphthyl, 1-(5,6,7,8-tetrahydro)naphthyl or 4-(1,2-dihydro)indenyl, pyridinyl, pyrimidyl, isoquinolinyl, 1,2,3,4-tetrahydroisoquinolinyl, benzofuranyl, benzothienyl, each of which is optionally substituted with up to three groups selected from halogen, C₁-C₆ alkyl, C₁-C₄ alkoxy, C₁-C₆ alkylthio, hydroxy, amino, mono or di(C₁-C₆)alkylamino, cyano, nitro, trifluoromethyl or trifluoromethoxy; or a carbocyclic group (“the X carbocyclic group”) containing from 3-7 members, up to two of which members are optionally hetero atoms selected from oxygen and nitrogen, where the X carbocyclic group is optionally substituted with one or more groups selected from halogen, alkoxy, mono- or dialkylamino, sulfonamide, azacycloalkyl, cycloalkylthio, alkylthio, phenylthio, or a heterocyclic group;
Y is lower alkyl having 1-8 carbon atoms optionally substituted with up to two groups selected from halogen, alkoxy, mono- or dialkylamino, sulfonamide, azacycloalkyl, cycloalkylthio, alkylthio, phenylthio, a heterocyclic group, —OR₄, —NR₅R₆, SR₇, or aryl; or a carbocyclic group (“the Y carbocyclic group”) having from 3-7 members atoms, where up to three of which members are optionally hetero atoms selected from oxygen and nitrogen and where any member of the Y carbocyclic group is optionally substituted with halogen, —OR₄, —NR₅R₆, SR₇, aryl or a heterocyclic group;
R₁ and R₄ are independently hydrogen, lower alkyl having 1-6 carbon atoms, or cycloalkyl having 3-7 carbon atoms, where each alkyl may be optionally substituted with —OR₄, or —NR₅R₆;
R₂, R₃, R₅ and R₆ are independently the same or different and represent hydrogen, lower alkyl optionally mono- or disubstituted with alkoxy, aryl, halogen, or mono- or di-lower alkyl; aryl or aryl(C₁-C₆)alkyl where each aryl is optionally substituted with up to three groups selected from halogen, hydroxy, C₁-C₆ alkyl, C₁-C₆ alkoxy, or mono- or di (C₁-C₆) alkylamino; cycloalkyl having 3-7 carbon atoms optionally mono or disubstituted with halogen, alkoxy, or mono- or di-lower alkyl; or —SO₂R₈;
R₇ is hydrogen, lower alkyl having 1-6 carbon atoms, or cycloalkyl having 3-7 carbon atoms; and

R₈ is lower alkyl having 1-6 carbon atoms, cycloalkyl having 3-7 carbon atoms, or optionally substituted phenyl;
which comprises treating a compound of the formula 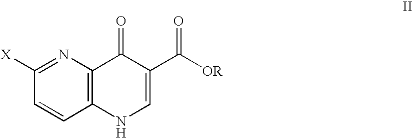
   wherein X, R₁, R₂, R₃, R₄, R₅, R₆, R₇ and R₈ are as defined above, and R is (C₁-C₆)alkyl with a primary amine of the formula 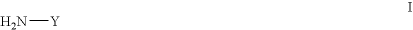
   wherein Y is as defined above.

The compounds which may be prepared by the process of the present invention can be described by general formulas I-III set forth above. In a further embodiment of the process of the present invention, in any of the aforesaid general formulas I-IV, X or Y may be —NR₂R₃ which is a heterocyclic group such as, for example, piperidine in the case where R₂ and R₃ together form a C₅-alkylene group. Further, R₂ and R₃ together may represent an alkylene or alkenylene group optionally containing up to two heteroatoms selected from nitrogen and oxygen. The resulting groups include imidazolyl, pyrrolidinyl, morpholinyl, piperazinyl, and piperidinyl.

Similarly, the —NR₅R₆ group in formula I above can also represent a heterocyclic group such as, for example, piperidine in the case where R₅ and R₆ together form a C₅-alkylene group. Further, R₅ and R₆ together may represent an alkylene or alkenylene group optionally containing up to two heteroatoms selected from nitrogen and oxygen. The resulting groups include imidazolyl, pyrrolidinyl, morpholinyl, piperazinyl, and piperidinyl.

Preferred compounds of formulas II and III are those where X represents (C₁-C₆) alkoxy, more preferably (C₁-C₃)alkoxy. Particularly preferred compounds of formulas II and III include methoxy or ethoxy as the X group.

For the process of the present invention other preferred compounds of formulas I-III include those where the Y is lower alkyl, e.g., methyl or ethyl, substituted with phenyl, pyridyl, or pyrimidinyl. A more preferred Y group is benzyl optionally substituted with halogen, (C₁-C₆)alkyl, (C₁-C₆)alkoxy, amino, or mono- or di(C₁-C₆) alkyl.

Where R₂ and R₃ in Formulas I-III represent optionally substituted aryl or aryl(C₁-C₆)alkyl, the aryl group is preferably phenyl, pyridyl, or pyrimidinyl and the alkyl groups are preferably methyl and ethyl. More preferred are benzyl and phenyl. Particularly preferred is benzyl.

Where X is optionally substituted C₁-C₆ alkyl, the alkyl group is preferably optionally substituted methyl, ethyl, or propyl. More preferred are perhalomethyl and trihaloethyl. Preferred halogens are fluorine. Particularly preferred is 2,2,2-trifluoroethyl.

X in formulas II and III may be an optionally substituted phenyl, naphthyl, 1-(5,6,7,8-tetrahydro)naphthyl, 4-(1,2-dihydro)indenyl, pyridinyl, pyrimidyl, isoquinolinyl, benzofuranyl, or benzothienyl group, or preferably a 1,2,3,4-tetrahydroisoquinolinyl group.

In addition to the compounds of formula III the process of the present invention encompasses the preparation of compounds of the formula 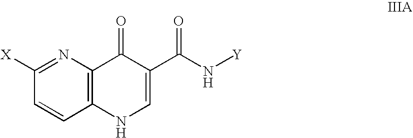
from compounds of the formula 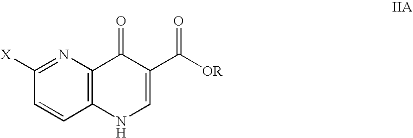
and
H₂N—Y IA
wherein substituent group X and substituent group Y when present either together or separately in any of the aforesaid general formulas IIA or IIIA are defined as follows:
X is:
(i) hydrogen, halogen, mono- or dialkylamino, alkoxy,
(ii) a group of the formula: 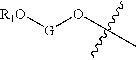
   where G is lower alkylene having 1-6 carbon atoms, or a cyclic group of the formula 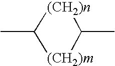
   where n is 0, 1, or 2, and m is an integer of from 1 to 5, with the proviso that the sum of n+m is not less than 1 or greater than 5; and R₁ is hydrogen, lower alkyl, or (C₃-C₇)cycloalkyl, where the alkyl or cycloalkyl is optionally substituted with halogen, lower alkoxy, or mono- or di(C₁-C₆)alkylamino;
(iii) a group of the formula: 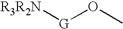
where G is as defined above for ii; and R₂ and R₃ independently represent hydrogen, lower alkyl having 1-6 carbon atoms, cycloalkyl having 3-7 carbon atoms, —SO₂ R₈ where R₈ is (C₁-C₆)alkyl, (C₃-C₇)cycloalkyl, or optionally substituted phenyl, or R₂ and R₃ together with the nitrogen atom to which they are attached form a heterocyclic moiety such as imidazolyl, pyrrolidinyl, morpholinyl, piperazinyl, or piperidinyl;
(iv) a group of the formula: 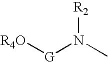
   where
R₂ is as defined above for iii;
R₄ is hydrogen, lower alkyl having 1-6 carbon atoms, or cycloalkyl having 3-7 carbon atoms, and may be optionally substituted with one or more (C₁-C₆)alkoxy or mono- or di(C₁-C₆)alkylamino groups; and
G is as defined above for ii;
(v) a group of the formula: 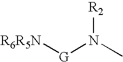
   where
R₂ and G are as defined above for iv and ii, respectively, and
R₅ and R₆ independently represent hydrogen, lower alkyl having 1-6 carbon atoms, cycloalkyl having 3-7 carbon atoms, —SO₂ R₈ where R₈ is (C₁-C₆)alkyl, (C₃-C₇)cycloalkyl, or optionally substituted phenyl, or
R₅ and R₆ together with the nitrogen atom to which they are attached form a heterocyclic moiety such as imidazolyl, pyrrolidinyl, morpholinyl, piperazinyl, or piperidinyl;
(vi) a group of the formula: 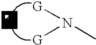
where G is as defined above for ii; or
(vii) a group of the formula: 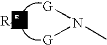
   where each G is as defined above for ii; and
Y is
(viii) lower alkyl having 1-8 carbon atoms or cycloalkyl having 3-7 carbon atoms, any of which may be optionally substituted with one or more hydroxy, halogen, (C₁-C₆)alkoxy, alkoxyalkoxy where each alkoxy is (C₁-C₆)alkoxy, (C₁-C₆) alkylthio, (C₃-C₇)cycloalkylthio, aryl, heteroaryl, or mono- or di(C₁-C₆)alkylamino groups;
(ix) a group of the formula: 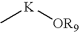
   where K is lower alkylene having 1-6 carbon atoms optionally substituted with (C₁-C₆)alkyl or alkylene, or a cyclic group of the formula 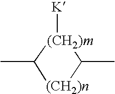
   where K′ independently represents hydrogen or (C₁-C₆) alkyl or alkylene, n is 0, 1, or 2, and m is an integer of from 1 to 5, with the proviso that the sum of n+m is not less than 1 or greater than 5; and R₉ is hydrogen, lower alkyl, or (C₃-C₇)cycloalkyl, where the alkyl or cycloalkyl is optionally substituted with halogen, lower alkoxy, or mono- or dialkylamino;
(x) a group of the formula: 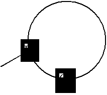
   where K is defined as above in ix;
(xi) a group of the formula: 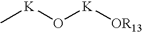
   where
K is as defined above for ix, and
R₁₃ is hydrogen, lower alkyl having 1-6 carbon atoms, or cycloalkyl having 3-7 carbon atoms, where the alkyl and cycloalkyl groups are optionally substituted with one or more (C₁-C₆)alkoxy or mono- or di(C₁-C₆)alkylamino groups; and
(xii) a group of the formula: 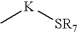
   where
K is as defined above for ix, and
R₇ is hydrogen, lower alkyl having 1-6 carbon atoms, or cycloalkyl having 3-7 carbon atoms; and
(xiii) a group of the formula: 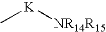
   where
K is as defined above for ix; and
R₁₄ and R₁₅ independently represent hydrogen, lower alkyl having 1-6 carbon atoms, cycloalkyl having 3-7 carbon atoms, —SO₂R₈ where R₈ is as defined above, or R₁₄ and R₁₃ together with the nitrogen atom to which they are attached form a heterocyclic moiety such as imidazolyl, pyrrolidinyl, morpholinyl, piperazinyl, or piperidinyl;
(xiv) a group of the formula: 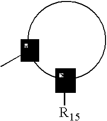
   where K and R₁₅ are as defined above in ix and xii, respectively;
(xv) a group of the formula: 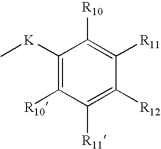
   where
K is as defined above for ix;
R₁₀ and R_{10′} are the same or different and are selected from hydrogen, (C₁-C₆)alkyl, halogen, hydroxy, lower alkoxy having 1-6 carbon atoms, or cycloalkoxy having 3-7 carbon atoms;
R₁₁, R_{11′}, and R₁₂ are the same or different and are selected from hydrogen, C₁-C₆ alkyl, halogen, hydroxy, —OR₄, —CR₇ (R₉)NR₅R₆, —CR₉(R₁₆) OR₄,
or R₁₁, and R₁₂ taken together with the atoms to which they are attached form a (hetero)cyclic ring; and

R₁₆ is hydrogen, lower alkyl having 1-6 carbon atoms, or cycloalkyl having 3-7 carbon atoms;
(xvi) a group of the formula: 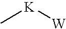
   where K is as defined above for ix; and W is heteroaryl;
(xvii) a group of the formula: 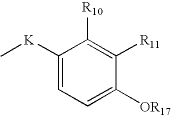
   where
K is as defined above for ix; R₁₀ and R₁ are as defined above for xv, and R₁₇ is hydrogen, lower alkyl, or (C₃-C₇)cycloalkyl, where the alkyl or cycloalkyl is optionally substituted with halogen, lower alkoxy, or mono- or di(C₁-C₆)alkylamino;
(xviii) a group of the formula: 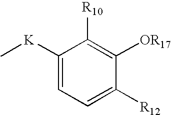
   where K, R₁₀, R₁₂, and R₁₇ are as defined above;
(xix) a group of the formula: 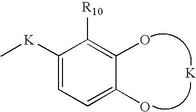
   where each K is independently as defined above for ix and R₁₀ is defined above;
(xx) a group of the formula: 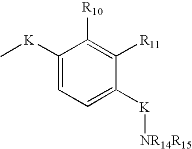
   where K, R₁₀, R₁₁, R₁₄, and R₁₅ are as defined above;
(xxi) a group of the formula: 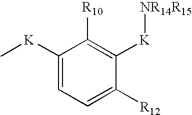
   where K, R₁₀, R₁₂, R₁₄, and R₁₅ are as defined above;
(xxii) pyrimidinyl(C₁-C₆)alkyl or pyridyl(C₁-C₆)alkyl; or
(xxiii) a group of the formula: 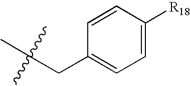
   where R₁₈ represents hydrogen, amino, mono-, or di(C₁-C₆)alkylamino, or C₁-C₆ alkyl optionally substituted with a R₁₉ where R₁₉ represents: 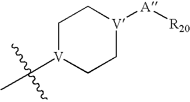
   where V and V′ are independently CH or nitrogen; A″ is C₁-C₆ alkylene; and
R₂₀ is phenyl, pyridyl, or pyrimidinyl, each of which is optionally mono-, di-, or trisubstituted independently with halogen, hydroxy, C₁-C₆ alkoxy, amino, or mono- or di(C₁-C₆)alkylamino.

Specific compounds made by the process of the invention include those having pyrimidinyl(C₁-C₆)alkyl Y groups, wherein Y is more specifically 2- and 4-pyrimidinylmethyl, or having pyridyl(C₁-C₆)alkyl Y groups, wherein Y is more specifically 2- and 4-pyridylmethyl.

Specific benzyl Y groups are those where R₁₈ is amino or a substituted methyl or ethyl group. More specific R₁₈ substituents are piperazin-1-yl or piperidin-1-yl substituted at the 4-position with a halogenated benzyl group.

Other specific benzyl Y groups are 4-[1-[4-(4-Fluorobenzyl)piperazinyl]methyl]benzyl and 4-[1-[4-(4-Fluorobenzyl)piperidinyl]methyl]benzyl.

Specific “X” groups in formulas IIIA and IIA are various quinolinyl, isoquinolinyl, tetrahydroquinolinyl, or tetrahydroisoquinolinyl groups, e.g., groups of the formulas:

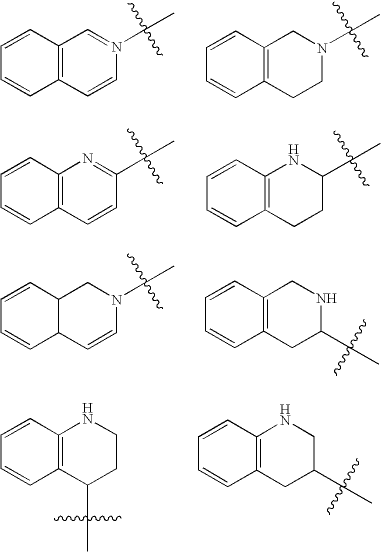

The following formulae represent specific compounds prepared by the process of the present invention: 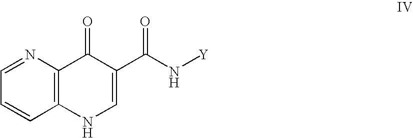
wherein Y is defined above. 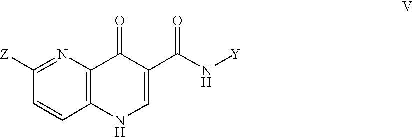
wherein Z represents halogen and Y is as defined above. 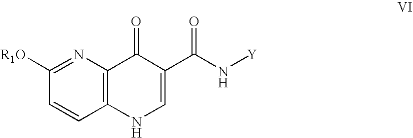
wherein R₁ and Y are defined above. 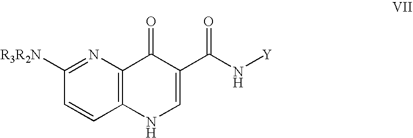
   wherein R₂, R₃, and Y are defined above. 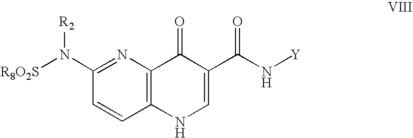
   wherein R₂, R₈, and Y are defined above. 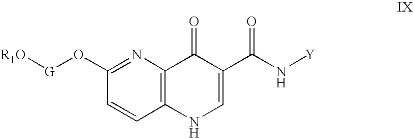
   wherein R₁, G and Y are defined above. 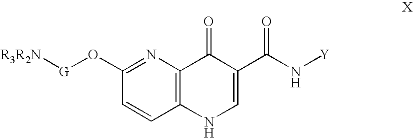
   wherein R₂, R₃, G, and Y are defined above. 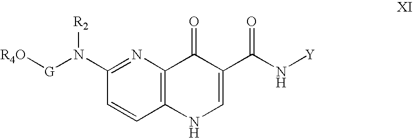
   wherein R₂, R₄, G, and Y are defined above. 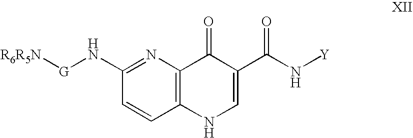
   wherein R₂, R₅, R₆, G, and Y are defined above. 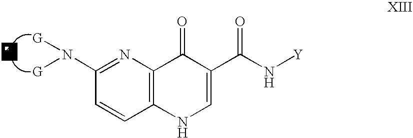
   wherein G and Y are defined above. 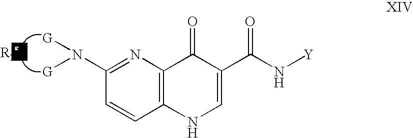
   wherein R₂, G, and Y are defined above. 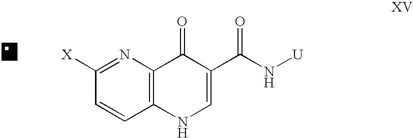
   wherein X is defined above and U is (C₁-C₆) lower alkyl or (C₁-C₆)cycloalkyl. 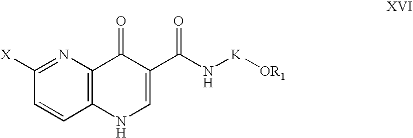
   wherein X, K, and R₁ are defined above. 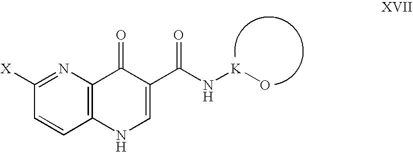
   wherein X and K are defined above. 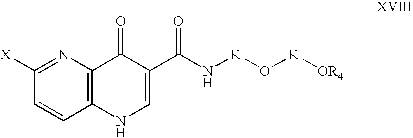
   wherein X, K, and R₄ are defined above. 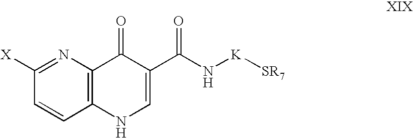
   wherein X, K, and R₇ are defined above. 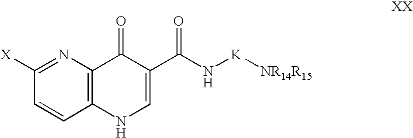
   wherein X, K, R₁₄, and R₁₅ are defined above. 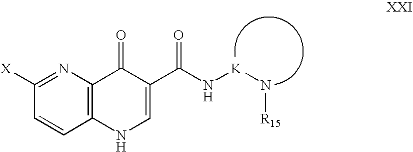
   wherein X, K, and R₁₅ are defined above. 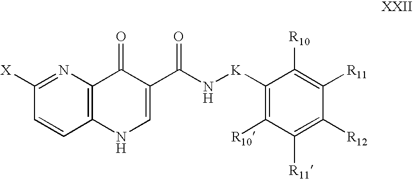
   wherein:
R₁₀, R₁₇ are the same or different and may be selected from hydrogen, (C₁-C₆)alkyl, halogen, hydroxy, lower alkoxy having 1-6 carbon atoms, or cycloalkoxy having 3-7 carbon atoms;
R₁₁, R_{11′}, and R₁₂ are the same or different and may be selected from hydrogen, (C₁-C₆)alkyl, halogen, hydroxy, —OR₄, —CR₇(R₉)NR₅R₆, —CR₇(R₉)OR₄; or
R₁₁, and R₁₂ taken together with the atoms to which they are attached form a (hetero)cyclic ring; and
R₉ is as defined above. 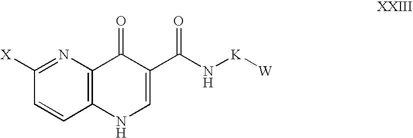
   wherein X and K are defined above; and W is heteroaryl. 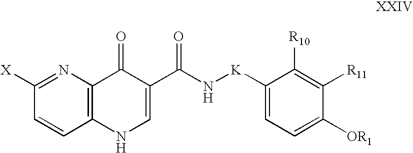
   wherein X, K, R₁, R₁₀, and R₁₁ are defined above. 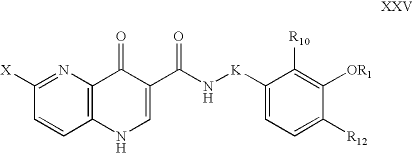
   wherein X, K, R₁, R₁₀, and R₁₂ are defined above. 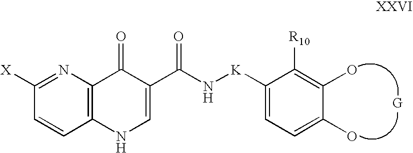
wherein X, K, R₁₀, and G are defined above. 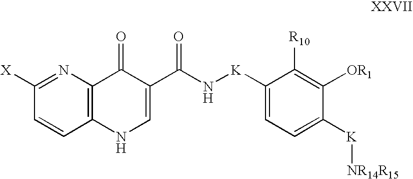
   wherein X, K, R₁₄, R₁₅, R₁₀, and R₁₁ are defined above.

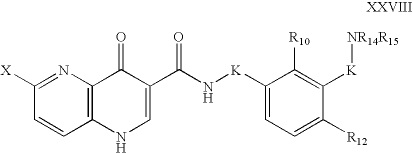

Specific compounds prepared by the process of the present invention are encompassed by the following formula: 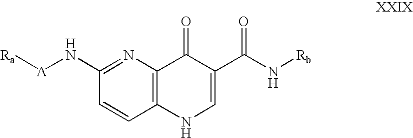
wherein A is C₁-C₆ alkylene;
Rₐ is phenyl optionally mono-, di-, or trisubstituted with halogen, lower alkyl, lower alkoxy, or mono- or di-C₁-C₆ alkylamino, or mono- or di-C₁-C₆ alkylamino lower alkyl; and
R_{b} is lower alkyl or lower cycloalkyl.

Other specific compounds of Formula XXIX made in accord with the invention are those wherein A is methylene, Rₐ is phenyl optionally substituted with methyl or ethyl, and R_{b} is lower alkyl. Still other specific compounds of Formula XXIX are those wherein A is methylene, Rₐ is phenyl and R_{b} is C₁-C₃ alkyl 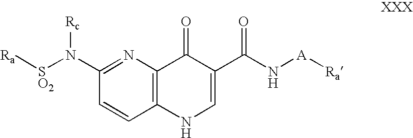
wherein A is C₁-C₆ alkylene;
Rₐ and Rₐ are independently phenyl groups optionally mono-, di-, or trisubstituted with halogen, lower alkyl, lower alkoxy, or mono- or di-C₁-C₆ alkylamino, or mono- or di-C₁-C₆ alkylamino lower alkyl; and
R_{c} is hydrogen or lower alkyl.

Specific compounds of Formula XXX made in accord with the invention are those where A is methylene, Rₐ and Rₐ are independently phenyl optionally substituted with methyl or ethyl, and R_{c} is lower alkyl. Other specific compounds of Formula XXX are those where A is methylene, Rₐ is phenyl substituted in the para position with lower alkyl, Rₐ is phenyl, and R_{c} is C₁-C₃ alkyl. 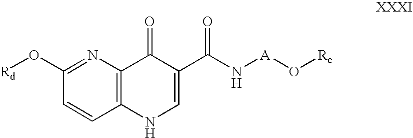
wherein A is C₁-C₆ alkylene;
R_{d} and Rₑ are independently lower alkyl groups.

Specific compounds of Formula XXXI made in accord with the invention are those where A is C₂-C₄ alkylene. Other specific compounds of Formula XXXI are those where A is C₂-C₄ alkylene, Rd is C₁-C₃ alkyl, and R_{d} is C₂-C₄ alkyl. 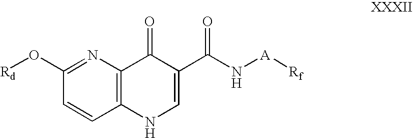
wherein A is C₁-C₆ alkylene;
R_{d} is lower alkyl; and
R_{f} is a group of the formula: 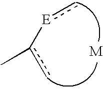
   where E is oxygen or nitrogen; and
M is C₁-C₃ alkylene or nitrogen.

Specific compounds of Formula XXXII made in accord with the invention are those where A is C₁-C₃ alkylene. Other specific compounds of Formula XXXII are those where A is C₂-C₄ alkylene, R_{d} is C₁-C₃ alkyl, and Rₑ is C₂-C₄ alkyl. Yet other specific compounds of Formula XXXII are those where A is C₂-C₄ alkylene, R_{d} is C₁-C₃ alkyl, Rₑ is C₂-C₄ alkyl, and E is nitrogen and M is methylene, E is oxygen and M is methylene or ethylene, or E and M are both nitrogen. Further specific compounds of Formula XXXII are those where R_{f} is furanyl, tetrahydrofuranyl, or imidazolyl. 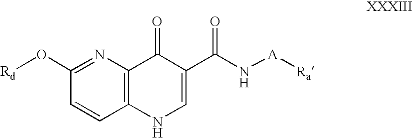
wherein
A is C₁-C₆ alkylene; Rd is lower alkyl optionally substituted with amino or mono- or di(C₁-C₆)alkylamino; and

Rₐ is phenyl optionally mono-, di-, or trisubstituted with halogen, lower alkyl, lower alkoxy, or mono- or di-C₁-C₆ alkylamino, or mono- or di-C₁-C₆ alkylamino lower alkyl.

Other specific compounds of Formula XXXIII are those where A is C₁-C₃ alkylene, Rₐ is phenyl optionally substituted with methyl or ethyl, and R_{d} is C₁-C₃ alkyl. Still other specific compounds of Formula XXXIII are where A is methylene, Rₐ is phenyl optionally substituted with methyl or ethyl, and R_{d} is C₃-C₆ alkyl. Other specific compounds of Formula XXXIII are those where Rₐ is phenyl substituted with mono- or di-(C₁-C₆) alkylamino lower alkyl 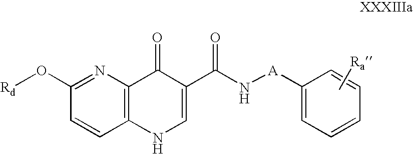
wherein
A is C₁-C₆ alkylene;
R_{d} is lower alkyl; and
Rₐ″ is phenyl, pyridyl, imidazolyl, pyrimidinyl, or pyrrolyl, each of which is optionally substituted with up to two groups selected from halogen, lower alkyl, lower alkoxy, mono- or di(C₁-C₆)alkylamino, or mono- or di-C₁-C₆ alkylamino lower alkyl.

Other specific compounds of Formula XXXIIla are those where Rₐ″ is imidazolyl and R_{d} is C₁-C₃ alkyl. Still other preferred compounds of Formula XXXIIla are where A is methylene, Rₐ″ is imidazolyl, and R_{d} is C₃-C₆ alkyl. 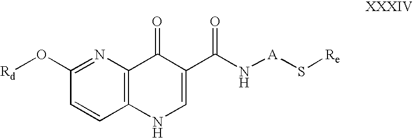
wherein
A is C₁-C₆ alkylene; and
R_{d} and Rₑ are independently lower alkyl groups.

Specific compounds of Formula XXXIV are those where A is C₁-C₃ alkylene. Other specific compounds of Formula XXXIV are those where A is C₁-C₃ alkylene, R_{d} is C₁-C₃ alkyl, and Rₑ is C₁-C₃ alkyl. 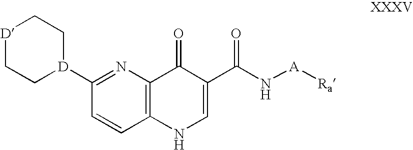
wherein
D is nitrogen or CH;
D′ is nitrogen or oxygen;
A is C₁-C₆ alkylene; and
Rₐ′ is phenyl, pyridyl, or thiazolyl, each of which is optionally mono-, di-, or trisubstituted with halogen, lower alkyl, lower alkoxy, or mono- or di-C₁-C₆ alkylamino, or mono- or di-C₁-C₆ alkylamino lower alkyl.

Specific compounds of Formula XXXV are those where A is C₁-C₃ alkylene, Rₐ is phenyl optionally substituted with lower alkyl or halogen, and D is nitrogen. Other specific compounds of Formula XXXV are where A is methylene, Rₐ is phenyl optionally substituted with lower alkyl or halogen, D is nitrogen, and D′ is oxygen. 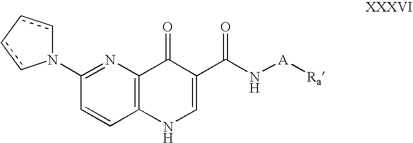
wherein
A is C₁-C₆ alkylene; and
Rₐ′ is hydrogen;
Rₐ is thienyl or phenyl, each of which is optionally mono-, di-, or trisubstituted with halogen, lower alkyl, lower alkoxy, or mono- or di-C₁-C₆ alkylamino, or mono- or di-C₁-C₆, alkylamino lower alkyl.

Specific compounds of Formula XXXVI are those where A is C₁-C₃ alkylene, and Rₐ is phenyl optionally substituted with lower alkyl or halogen. Other specific compounds of Formula XXXVI are where A is methylene, Rₐ is phenyl optionally substituted with lower alkyl, lower alkoxy or halogen. 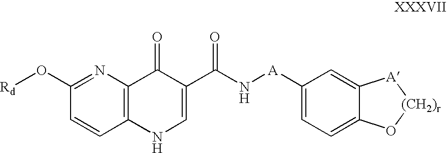
wherein
A is C₁-C₆ alkylene; and
R_{d} is lower alkyl;
A′ represents oxygen or methylene; and
r is an integer of from 1-3.

Specific compounds of Formula XXXVII are those where A is C₁-C₃ alkylene. Other specific compounds of Formula XXXVII are those where A is C₁-C₃ alkylene, and R_{d} is C₁-C₃ alkyl. 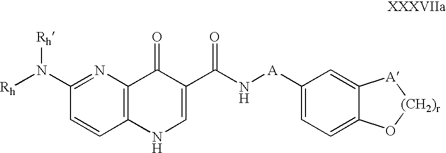
wherein A is C₁-C₆ alkylene; and
Rₕ and Rₕ′ are independently hydrogen or lower alkyl, where each alkyl is optionally substituted with lower alkoxy;
A′ represents oxygen or methylene; and
r is an integer of from 1-3.

Specific compounds of Formula XXXVIIa are those where A is C₁-C₃ alkylene. Other specific compounds of Formula XXXVIIa are those where A is C₁-C₃ alkylene, and Rₕ is C₁-C₃ alkyl. 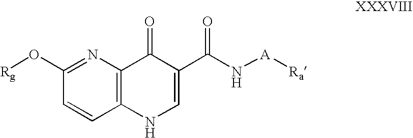
wherein A is C₁-C₆ alkylene;
R₉ is lower alkoxy lower alkyl; and
Rₐ′ is phenyl optionally mono-, di-, or trisubstituted with halogen, lower alkyl, lower alkoxy, or mono- or di-C₁-C₆ alkylamino, or mono- or di-C₁-C₆ alkylamino lower alkyl. 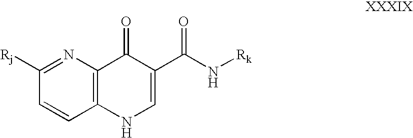
   wherein
Rⱼ is halogen or lower alkoxy; and
Rₖ is lower alkyl or cycloalkyl each of which is optionally substituted with hydroxy, lower alkyl, or lower alkoxy; or
Rₖ is phenyl (C₁-C₆) alkyl where the phenyl group is optionally mono-, di-, or trisubstituted with halogen, lower alkyl, lower alkoxy, or mono- or di-C₁-C₆ alkylamino, or mono- or di-C₁-C₆ alkylamino lower alkyl. 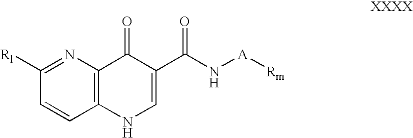
   wherein
A is C₁-C₆ alkylene;
Rₗ is lower alkoxy, benzyloxy, lower alkoxy lower alkoxy, amino, or mono- or di-(C₁-C₆)alkylamino; and
Rₘ is pyranyl, dihydropyranyl, tetrahydropyranyl, or hexahydropyranyl, pyridine, dihydropyridine, tetrahydropyridine, or piperidine.

Specific compounds of Formula XXXX are those where Rₗ is lower alkoxy and Rₘ is tetrahydropyranyl. 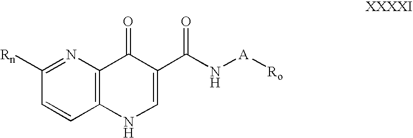
wherein
A is C₁-C₆ alkylene;
Rₙ is lower alkoxy, benzyl, or a group of the formula: 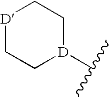
   where
D is nitrogen or CH; and
D′ is nitrogen or oxygen; and
Rₒ is pyranyl, 2- or 3-thienyl; or
Rₒ is 2-, 4-, or 5-thiazolyl or 2-, 4-, or 5-imidazolyl, each of which may be optionally substituted with lower alkyl 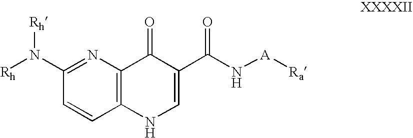
   wherein
A is C₁-C₆ alkylene;
Rₕ and Rₕ′ are independently hydrogen or lower alkyl, where each lower alkyl is optionally substituted with lower alkoxy; and
Rₐ′ is phenyl optionally mono-, di-, or trisubstituted with halogen, lower alkyl, lower alkoxy, or mono- or di-C₁-C₆ alkylamino, or mono- or di-C₁-C₆ alkylamino lower alkyl; or
Rₐ′ is thienyl optionally substituted with lower alkyl 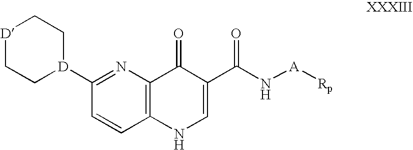
   wherein A is C₁-C₆ alkylene;
D is nitrogen or CH;
D′ is nitrogen or oxygen; and
Rₚ is lower alkyl or lower alkyl optionally substituted with lower alkoxy. 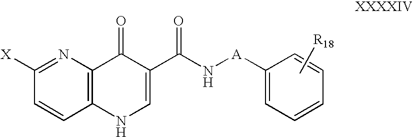
   wherein
A is C₁-C₆ alkylene;
X is defined as above for Formula IV; and R₁₈ is
(i) amino or mono- or di(C₁-C₆)alkylamino; or
(ii) lower alkyl optionally substituted with 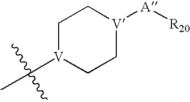
   where
V and V′ are independently CH or nitrogen;
A″ is C₁-C₆ alkylene; and
R₂₀ is phenyl, pyridyl, or pyrimidinyl, each of which is optionally mono-, di-, or trisubstituted independently with halogen, hydroxy, C₁-C₆ alkoxy, amino, or mono- or di(C₁-C₆)alkylamino.

Specific compounds of Formula XXXXIV are those where V is nitrogen and X is C₁-C₆ alkoxy or C₁-C₆ alkyl optionally substituted with up to three halogen atoms. Other specific compounds of XXXXIV are those where V and V′ are nitrogen; X is C₁-C₃ alkoxy or C₁-C₃ alkyl optionally substituted with up to three halogen atoms; A″ is methylene or ethylene; and R₂₀ is halogenated phenyl. A specific R₂₀ group is 4-fluorophenyl. Yet other specific compounds of XXXXIV are those where X is 2,2,2-trifluoroethyl; V and V′ are nitrogen; R₂₀ is halogenated phenyl; and A and A″ are methylene or ethylene.

In certain situations, compounds of Formulas II and III may contain one or more asymmetric carbon atoms, so that the compounds can exist in different stereoisomeric forms. These compounds can be, for example, racemates or optically active forms. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral HPLC column.

Representative compounds which are encompassed by Formula III, and may be prepared by the process of the present invention include, but are not limited to, the compounds in Table I and their pharmaceutically acceptable acid and base addition salts. In addition, if the compound of the invention is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds.

Non-toxic pharmaceutical salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)n-COOH where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

The process of the present invention also encompasses the acylated prodrugs of the compounds of Formula III. Those skilled in the art will recognize various synthetic methodologies which may be employed to prepare non-toxic pharmaceutically acceptable addition salts and acylated prodrugs of the compounds encompassed by Formula III.

By lower alkyl in the present invention is meant straight or branched chain alkyl groups having 1-6 carbon atoms, such as, for example, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl.

By cycloalkyl in the present invention is meant cycloalkyl groups having 3-7 atoms such as, for example cyclopropyl, cyclobutyl, cyclopentyl, cyclohexyl, and cycloheptyl.

By aryl is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl), which is optionally mono-, di-, or trisubstituted with, e.g., halogen, lower alkyl, lower alkoxy, lower alkylthio, trifluoromethyl, lower acyloxy, aryl, heteroaryl, and hydroxy.

By lower alkoxy in the present invention is meant straight or branched chain alkoxy groups having 1-6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

By cycloalkoxy in the present invention is meant cycloalkylalkoxy groups having 3-7 carbon atoms where cycloalkyl is defined above.

By halogen in the present invention is meant fluorine, bromine, chlorine, and iodine.

By heteroaryl (aromatic heterocycle) in the present invention is meant one or more aromatic ring systems of 5-, 6-, or 7-membered rings containing at least one and up to four hetero atoms selected from nitrogen, oxygen, or sulfur. Such heteroaryl groups include, for example, thienyl, furanyl, thiazolyl, imidazolyl, (is)oxazolyl, pyridyl, pyrimidinyl, (iso)quinolinyl, naphthyridinyl, benzimidazolyl, and benzoxazolyl.

Specific examples of heteroaryl groups are the following: 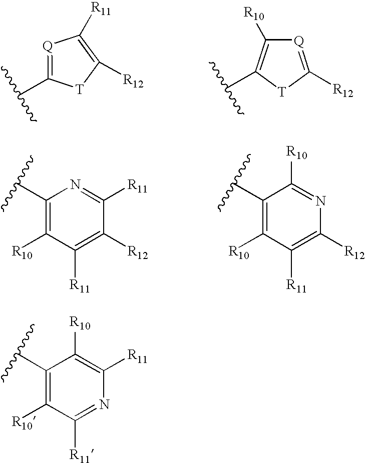
wherein Q is nitrogen or —CR₉;
T is —NR₇, oxygen, or sulfur; and
R₉, R₁₀, R₁₀′, R₁₁, R₁₁′, R₁₂ are as defined above,
where Y represents a carbocyclic group, it is attached to the amide nitrogen by a single bond. The result is an amide of the formula: 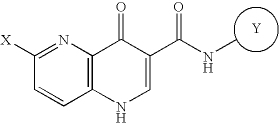
   where X is defined as above and 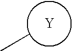
   represents the Y carbocyclic group.

Where X is a carbocyclic group, such moiety or group includes both aromatic heterocycles (heteroaryl), unsaturated heterocylic ring systems, and saturated heterocyclic ring systems. Examples of such groups are imidazolyl, pyrrolidinyl, morpholinyl, piperazinyl, or piperidinyl. Specific X carbocyclic groups are linked to the parent naphthyridine moiety by a nitrogen atom in the X carbocyclic group. Thus, for example, when pyrrolidinyl is the X carbocyclic group, it is specifically a 1-pyrrolidinyl group of the formula:

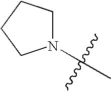

Where Y is a carbocyclic group, such moiety or group includes both aromatic heterocycles (heteroaryl groups), unsaturated heterocylic ring systems, and saturated heterocyclic ring systems. Examples of such groups are imidazolyl, pyrrolidinyl, morpholinyl, piperazinyl, or piperidinyl. Specific Y carbocyclic groups are linked to the parent naphthyridine carboxamide group by a nitrogen atom in the Y carbocyclic group. Thus, for example, when piperidinyl is the Y carbocyclic group, it is specifically a 1-piperidinyl group of the formula:

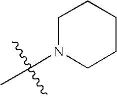

By “optionally substituted phenyl” as used herein is meant phenyl groups that are unsubstituted or substituted with up to 3 groups selected independently from halogen, hydroxy, lower alkyl, lower alkoxy, trifluoromethyl, and mono- or di-lower alkylamino.

Representative compounds that may be prepared by the process of the present invention are shown below in Table 1.

TABLE 1

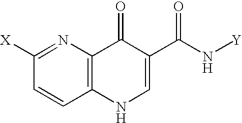

| | X | Y |
|---|---|---|
| 1. | C₆H₅CH₂NH— | —CH₂CH₂CH₂CH₃ |
| 2. | p-CH₃C₆H₄SO₂N(CH₃)— | —CH₂C₆H₅ |
| 3. | CH₃CH₂O— | —CH₂CH₂CH₂OCH(CH₃)₂ |
| 4. | CH₃CH₂O— | 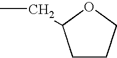 |
| 5. | CH₃O— | —CH₂CH₂SCH₂CH₃ |
| 6. | CH₃CH₂O— | 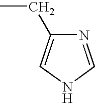 |
| 7. | O(CH₂CH₂)₂N— | —CH₂C₆H₄F-o |
| 8. | (CH₂CH₂CH₂CH₂)N— | —CH₂C₆H₄OCH₃-p |
| 9. | CH₃CH₂O— | 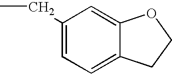 |
| 10. | CH₃CH₂O— | —CH₂C₆H₄CH₂NHCH₃-p |
| 11. | CH₃CH₂O— | —CH₂C₆H₅ |

### DETAILED DESCRIPTION OF THE INVENTION

The process of the present invention and the preparation of the compounds of the present invention are illustrated in Scheme 1. The preparation of the compound of Formula III from the compound of Formula II is described in Scheme 1 and the discussion that follows, wherein, unless otherwise indicated, X and Y are as defined above.

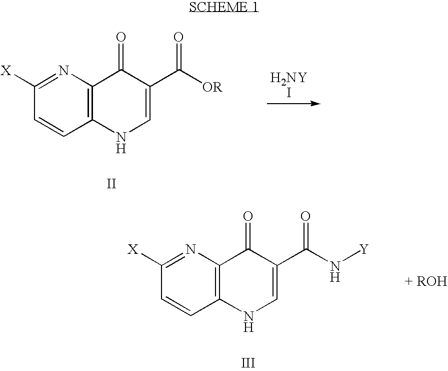

Overall the synthetic sequence of the scheme involves a single step which is direct reaction of the ester having structure II with primary amine I to form the carboxyamide having structure III.

In Scheme 1 the ester having structure II is treated with a primary amine, preferably an excess of primary amine, and heated to form carboxyamide III directly. Scheme 1 may be carried out without a solvent other than the amine but the use of a solvent, especially a polar solvent, is preferred. Preferred solvents include amide solvents such as dimethylacetamide (DMAc), dimethylformamide (DMF) or N-methylpyrollidone (NMP), or a sulfoxide solvent, such as dimethylsulfoxide (DMSO). Scheme 1 is carried out by heating the ester of formula II from about 90° C. to approximately the reflux temperature of the solvent, preferably to about 150° C., more preferably to about 105° C. to about 1100 C for about 1 hour to about 24 hours, with about 14 hours preferred. The process of Scheme 1 is preferably carried out under an inert atmosphere such as nitrogen or argon although this is not essential. The solution is cooled to about 5° C. to about 35° C., with about 22° C. preferred. The solution is then poured into water and the precipitated solid washed filtered and dried and optionally recrystallized. In a preferred variation, the reaction mixture is filtered and the residue is washed with solvent with the washings added to the filtrate. While maintaining the filtrate below about 35° C. equal quantities of acetone and water are added and the mixture is acidified to about pH 3 with an acid, preferably HCl to form a slurry which is then filtered dried and optionally recrystallized. Other variants on this general procedure will be evident to those skilled in the art.

The present invention is illustrated by the following examples, but it is not limited to the details thereof.

### EXAMPLE 1

N-benzyl-6-ethoxy-4-oxo-1,4-dihydro-1,5-naphthyridine-3-carboxamide

A mixture of 6-ethoxy-4-oxo-1,4-dihydro-1,5-naphthyridine-3-carboxylic acid ethyl ester (0.964 g, 4.1 mM) and benzylamine (1.97 g, about 5 equivalents) in 10 mL of DMAc was heated at 150° C. overnight. The clear solution was cooled to room temperature and poured into 50 mL water. The precipitated solid was filtered and dried. HPLC showed about 9% of the title product was present with the remainder unreacted starting material.

### EXAMPLE 2

N-benzyl-6-ethoxy-4-oxo-1,4-dihydro-1.5-naphthyridine-3-carboxamide—DMSO Solvent

A slurry of 6-ethoxy-4-oxo-1,4-dihydro-1,5-naphthyridine-3-carboxylic acid ethyl ester (10 g, 0.038 M) and 50 ml dimethylsulfoxide (DMSO) were heated to 105°-110° C. Benzylamine (12.5 g, 0.12 M −15.0 g, 0.14 M) was added to the heated slurry. The addition flask was rinsed with 5 ml DMSO which was also added to the slurry. The heated reaction mixture was stirred for 2-6 hours and then cooled to room temperature. The reaction mixture was filtered and the residue rinsed with 5 ml DMSO. Acetone (25 mL) and water 25 mL were added to the filtrate while maintaining it at a temperature below 35° C. The acidity of the mixture was adjusted to pH 3 with 6-8 mL of concentrated HCl. The slurry was diluted with 23 mL water and cooled to about 50 C. The product was collected by filtration and washed with 100 mL water and then dried under vacuum at about 70° C. to give an average of 12 g (97.4% yield) of the title compound.

The product was recrystallized by dissolving the solid in 120 mL acetic acid at temperatures greater than 90° C. and filtering the resultant solution. The filtrate was cooled to about 60° C. and then diluted with 32-50 mL of water having a temperature of approximately 55° C. The filtrate was slowly cooled to about 3° C. The product was collected and washed with 60 mL water and dried under vacuum at about 70° C. to recover an average of 8.35 g (69.6% recovery) for an average overall yield of 67.5%. The product was milled through a 0.05 round hole screen.

### EXAMPLE 3

N-benzyl-6-ethoxy-4-oxo-1,4-dihydro-1,5-naphthyridine-3-carboxamide—DMF Solvent

A slurry of 6-ethoxy-4-oxo-1,4-dihydro-1,5-naphthyridine-3-carboxylic acid ethyl ester (10 g, 0.038 M) and 50 ml dimethylformamide (DMF) were heated to 1050-1100 C. Benzylamine (17 g, 0.16 M) was added to the heated slurry. The addition flask was rinsed with 5 ml DMSO which was also added to the slurry. The heated reaction mixture was stirred at least 14 hours and then cooled to about 40° C. The reaction mixture was filtered and the residue rinsed with 5 ml DMF. Water (120 mL) was added to the filtrate while maintaining it at a temperature below 45° C. The acidity of the mixture was adjusted to pH 4 with about 15 mL of concentrated HCl. The mixture was cooled to about 5° C. The product was collected by filtration and washed with 40 mL water and then dried under vacuum at about 70° C. to give 8.9 g (72.2% yield) of the title compound.

The product was recrystallized by dissolving the solid in 89 mL acetic acid at temperatures greater than 90° C. and filtering the resultant solution. The filtrate was cooled to about 55° C. and then diluted with 22 mL of water having a temperature of approximately 50° C. The filtrate was slowly cooled to about 3° C. The product was collected by filtration and washed with 53 mL water and dried under vacuum at about 70° C. to recover 6.3 g (70.8% recovery) for an average overall yield of 51%. The product was milled through a 0.05 round hole screen.

1. A process of preparing a compound of the formula 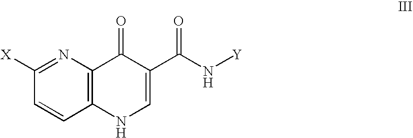
wherein X is hydrogen, halogen, —OR₁, C₁-C₆ alkyl optionally substituted with up to three groups selected independently from halogen and hydroxy, or —NR₂R₃; phenyl, naphthyl, 1-(5,6,7,8-tetrahydro)naphthyl or 4-(1,2-dihydro)indenyl, pyridinyl, pyrimidyl, isoquinolinyl, 1,2,3,4-tetrahydroisoquinolinyl, benzofuranyl, benzothienyl, each of which is optionally substituted with up to three groups selected from halogen, C₁-C₆ alkyl, C₁-C₄ alkoxy, C₁-C₆ alkylthio, hydroxy, amino, mono or di(C₁-C₆)alkylamino, cyano, nitro, trifluoromethyl or trifluoromethoxy; or
a carbocyclic group containing from 3-7 members, up to two of which members are optionally hetero atoms selected from oxygen and nitrogen, where the X carbocyclic group is optionally substituted with one or more groups selected from halogen, alkoxy, mono- or dialkylamino, sulfonamide, azacycloalkyl, cycloalkylthio, alkylthio, phenylthio, or a heterocyclic group;
Y is lower alkyl having 1-8 carbon atoms optionally substituted with up to two groups selected from halogen, alkoxy, mono- or dialkylamino, sulfonamide, azacycloalkyl, cycloalkylthio, alkylthio, phenylthio, a heterocyclic group, —OR₄, —NR₅R₆, SR₇, or aryl; or
a carbocyclic group having from 3-7 members atoms, where up to three of which members are optionally hetero atoms selected from oxygen and nitrogen and where any member of the Y carbocyclic group is optionally substituted with halogen, —OR₄, —NR₅R₆, SR₇, aryl or a heterocyclic group;
R₁ and R₄ are independently hydrogen, lower alkyl having 1-6 carbon atoms, or cycloalkyl having 3-7 carbon atoms, where each alkyl may be optionally substituted with —OR₄, or —NR₅R₆;
R₂, R₃, R₅ and R₆ are independently the same or different and represent hydrogen, lower alkyl optionally mono- or disubstituted with alkoxy, aryl, halogen, or mono- or di-lower alkyl; aryl or aryl(C₁-C₆)alkyl where each aryl is optionally substituted with up to three groups selected from halogen, hydroxy, C₁-C₆ alkyl, C₁-C₆ alkoxy, or mono- or di(C₁-C₆)alkylamino; cycloalkyl having 3-7 carbon atoms optionally mono or disubstituted with halogen, alkoxy, or mono- or di-lower alkyl; or —SO₂R₈;
R₇ is hydrogen, lower alkyl having 1-6 carbon atoms, or cycloalkyl having 3-7 carbon atoms; and
R₈ is lower alkyl having 1-6 carbon atoms, cycloalkyl having 3-7 carbon atoms, or optionally substituted phenyl;
said process comprising the step of heating a compound of the formula 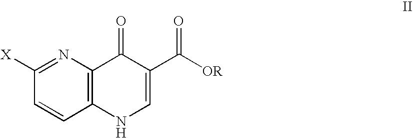
wherein R is (C₁-C₆)alkyl, in the presence of a primary amine of the formula H₂N—Y (I).

2. The process of claim 1 wherein the compound of formula III is prepared by heating the compound of formula II in the presence of an excess of the amine of formula I.

3. The process of claim 2 wherein the compound of formula III is prepared by heating the compound of formula II in the presence of an excess of the amine of formula I in a polar solvent.

4. The process of claim 3 wherein the compound of formula III is prepared by heating the compound of formula II in the presence of an excess of the amine of formula I in a polar solvent selected from amide solvents and sulfoxide solvents.

5. The process of claim 4 wherein the compound of formula III is prepared by heating the compound of formula II in the presence of an excess of the amine of formula I in a polar solvent selected from dimethylformamide, dimethylacetamide, N-methylpyrollidone or dimethylsulfoxide.

6. The process of claim 5 wherein the compound of formula III is prepared by heating the compound of formula II in the presence of an excess of the amine of formula I at a temperature of about 90° C. to about 150° C.

7. A process of preparing a compound of the formula 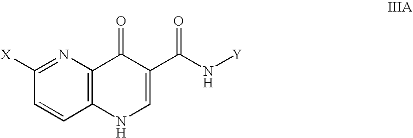
wherein X is
(i) hydrogen, halogen, mono- or dialkylamino, alkoxy,
(ii) a group of the formula: 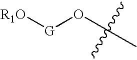
where G is lower alkylene having 1-6 carbon atoms, or a cyclic group of the formula 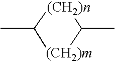
where n is 0, 1, or 2, and m is an integer of from 1 to 5, with the proviso that the sum of n+m is not less than 1 or greater than 5; and R₁ is hydrogen, lower alkyl, or (C₃-C₇)cycloalkyl, where the alkyl or cycloalkyl is optionally substituted with halogen, lower alkoxy, or mono- or di(C₁-C₆)alkylamino;
(iii) a group of the formula: 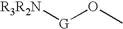
where G is as defined above for ii; and R₂ and R₃ independently represent hydrogen, lower alkyl having 1-6 carbon atoms, cycloalkyl having 3-7 carbon atoms, —SO₂R₈ where R₈ is (C₁-C₆)alkyl, (C₃-C₇)cycloalkyl, or optionally substituted phenyl, or R₂ and R₃ together with the nitrogen atom to which they are attached form a heterocyclic moiety selected from imidazolyl, pyrrolidinyl, morpholinyl, piperazinyl, or piperidinyl;
(iv) a group of the formula: 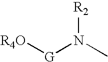
where
R₂ is as defined above for iii;
R₄ is hydrogen, lower alkyl having 1-6 carbon atoms, or cycloalkyl having 3-7 carbon atoms, and may be optionally substituted with one or more (C₁-C₆)alkoxy or mono- or di(C₁-C₆)alkylamino groups; and
G is as defined above for ii;
(v) a group of the formula: 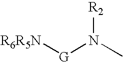
where
R₂ and G are as defined above for iv and ii, respectively, and R₅ and R₆ independently represent hydrogen, lower alkyl having 1-6 carbon atoms, cycloalkyl having 3-7 carbon atoms, —SO₂R₈ where R₆ is (C₁-C₆)alkyl, (C₃-C₇)cycloalkyl, or optionally substituted phenyl, or R₅ and R₆ together with the nitrogen atom to which they are attached form a heterocyclic moiety;
(vi) a group of the formula: 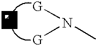
where G is as defined above for ii; or
(vii) a group of the formula: 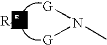
where each G is as defined above for ii; and
Y is
(viii) lower alkyl having 1-8 carbon atoms or cycloalkyl having 3-7 carbon atoms, any of which may be optionally substituted with one or more hydroxy, halogen, (C₁-C₆)alkoxy, alkoxyalkoxy where each alkoxy is (C₁-C₆)alkoxy, (C₁-C₆) alkylthio, (C₃-C₇)cycloalkylthio, aryl, heteroaryl, or mono- or di(C₁-C₆)alkylamino groups;
(ix) a group of the formula: 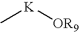
where K is lower alkylene having 1-6 carbon atoms optionally substituted with (C₁-C₆)alkyl or alkylene, or a cyclic group of the formula 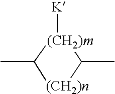
where K′ independently represents hydrogen or (C₁-C₆) alkyl or alkylene, n is 0, 1, or 2, and m is an integer of from 1 to 5, with the proviso that the sum of n+m is not less than 1 or greater than 5; and
R₉ is hydrogen, lower alkyl, or (C₃-C₇)cycloalkyl, where the alkyl or cycloalkyl is optionally substituted with halogen, lower alkoxy, or mono- or dialkylamino;
(x) a group of the formula: 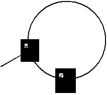
where K is defined as above in ix;
(xi) a group of the formula: 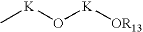
where
K is as defined above for ix, and
R₁₃ is hydrogen, lower alkyl having 1-6 carbon atoms, or cycloalkyl having 3-7 carbon atoms, where the alkyl and cycloalkyl groups are optionally substituted with one or more (C₁-C₆)alkoxy or mono- or di(C₁-C₆)alkylamino groups; and
(xii) a group of the formula: 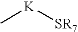
where
K is as defined above for ix, and
R₇ is hydrogen, lower alkyl having 1-6 carbon atoms, or cycloalkyl having 3-7 carbon atoms; and
(xiii) a group of the formula: 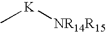
where
K is as defined above for ix; and
R₁₄ and R₁₅ independently represent hydrogen, lower alkyl having 1-6 carbon atoms, cycloalkyl having 3-7 carbon atoms, —SO₂R₈ where R₈ is as defined above, or
R₁₄ and R₁₃ together with the nitrogen atom to which they are attached form a heterocyclic moiety;
(xiv) a group of the formula: 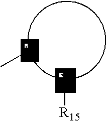
where K and R₁₅ are as defined above in ix and xii, respectively;
(xv) a group of the formula: 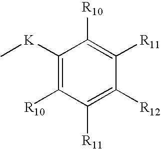
where
K is as defined above for ix;
R₁₀ and R₁₀, are the same or different and are selected from hydrogen, (C₁-C₆)alkyl, halogen, hydroxy, lower alkoxy having 1-6 carbon atoms, or cycloalkoxy having 3-7 carbon atoms;
R₁₁, R_{11′}, and R₁₂ are the same or different and are selected from hydrogen, C₁-C₆ alkyl, halogen, hydroxy, —OR₄, —CR₇(R₉)NR₅R₆, —CR₉(R₁₆)OR₄,
or R₁₁ and R₁₂ taken together with the atoms to which they are attached form a (hetero)cyclic ring; and
R₁₆ is hydrogen, lower alkyl having 1-6 carbon atoms, or cycloalkyl having 3-7 carbon atoms;
(xvi) a group of the formula: 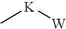
where K is as defined above for ix; and W is heteroaryl;
(xvii) a group of the formula: 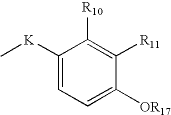
where
K is as defined above for ix; R₁₀ and R₁₁ are as defined above for xv, and R₁₇ is hydrogen, lower alkyl, or (C₃-C₇)cycloalkyl, where the alkyl or cycloalkyl is optionally substituted with halogen, lower alkoxy, or mono- or di(C₁-C₆)alkylamino;
(xviii) a group of the formula: 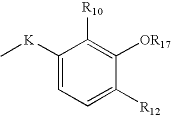
where K, R₁₀, R₁₂, and R₁₇ are as defined above;
(xix) a group of the formula: 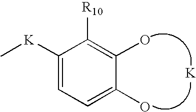
where each K is independently as defined above for ix and R₁₀ is defined above;
(xx) a group of the formula: 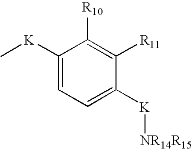
where K, R₁₀, R₁₁, R₁₄, and R₁₅ are as defined above;
(xxi) a group of the formula: 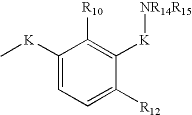
where K, R₁₀, R₁₂, R₁₄, and R₁₅ are as defined above;
(xxii) pyrimidinyl (C₁-C₆)alkyl or pyridyl (C₁-C₆)alkyl; or
(xxiii) a group of the formula: 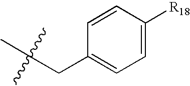
where R₁₈ represents hydrogen, amino, mono-, or di(C₁-C₆)alkylamino, or C₁-C₆ alkyl optionally substituted with a R₁₉ where R₁₉ represents: 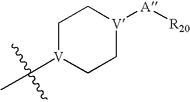
where V and V′ are independently CH or nitrogen; A″ is C₁-C₆ alkylene; and
R₂₀ is phenyl, pyridyl, or pyrimidinyl, each of which is optionally mono-, di-, or trisubstituted independently with halogen, hydroxy, C₁-C₆ alkoxy, amino, or mono- or di(C₁-C₆)alkylamino;
which comprises heating a compound of the formula 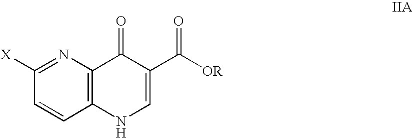
wherein R is (C₁-C₆)alkyl, in the presence of a primary amine of the formula
H₂N—Y (IA).

8. The process of claim 7 wherein the compound of formula IIIA is prepared by heating the compound of formula IIA in the presence of an excess of the amine of formula IA.

9. The process of claim 8 wherein the compound of formula IIIA is prepared by heating the compound of formula IIA in the presence of an excess of the amine of formula IA in a polar solvent.

10. The process of claim 9 wherein the compound of formula IIIA is prepared by heating the compound of formula IIA in the presence of an excess of the amine of formula IA in a polar solvent selected from amide solvents and sulfoxide solvents.

11. The process of claim 10 wherein the compound of formula IIIA is prepared by heating the compound of formula IIA in the presence of an excess of the amine of formula IA in a polar solvent selected from dimethylformamide, dimethylacetamide, N-methylpyrollidone or dimethylsulfoxide.

12. The process of claim 11 wherein the compound of formula IIIA is prepared by heating the compound of formula IIA in the presence of an excess of the amine of formula IA at a temperature of about 90° C. to about 150° C.
